(19) United States
(12) Patent Application Publication
(10) Pub. No.: US 2008/0014245 A1
(43) Pub. Date: Jan. 17, 2008

(54) Drug-eluting implantable medical device with free radical scavenger for protecting drugs during sterilization and related method

(76) Inventors: Stephen Pacetti, San Jose, CA (US); Jessica Renee DesNoyer, San Jose, CA (US); Gina Zhang, Temecula, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/487,253

(22) Filed: Jul. 14, 2006

(51) Int. Cl. A61F 2/02; C08L 75/06

(52) U.S. Cl. 424/426; 525/440.04

(57) ABSTRACT

The current invention relates to devices and methods for protecting drugs coated on implantable medical devices, in particular drug-eluting stents, involving the inclusion of free or, preferably at present, polymer-bound stable nitroxides in a coating layer on the device that is either the same layer that contains the drug or that is located between the drug-containing layer and the source of free electrons used to sterilize the device.

### FIELD

This invention relates to the field of organic chemistry, polymer chemistry, material science and medical devices.

### BACKGROUND

Devices intended to be implanted in the body of a patient must be sterilized prior to implantation. While many usable sterilization techniques exist, for all intents and purposes three dominate the field: ethylene oxide sterilization, gamma radiation sterilization and e-beam sterilization.

Ethylene oxide is one of the oldest methods of sterilization, having been in use for over 60 years, and remains an effective sterilization procedure that works well with many products. It sterilizes through an alkalization reaction that prevents organisms from reproducing. It is, however, time-consuming. First, it generally requires a conditioning step which constitutes placing the product to be sterilized in a high humidity environment to render it more penetrable by the ethylene oxide gas. The sterilization step itself can take several hours. Once completed the product must be “aerated” to allow residual ethylene oxide to dissipate. In addition ethylene oxide sterilization often requires inclusion of an indicator, a specially contaminated material that is placed in the sterilization chamber with the product to be sterilized and then tested for sterility after removal to insure success of the process.

Gamma irradiation sterilization, which once accounted for a mere 5% of the sterilization market currently captures up to 50%. It involves exposure of a product to be sterilized to Cobalt-60, a radioactive isotope that emits gamma rays. Gamma irradiation sterilizes by interacting with the electrons forming chemical bonds resulting in breakage of the bonds and disintegration of the molecule. The gamma irradiation procedure is repeatable and relatively easy to use. The procedure is, however, somewhat inflexible in that gamma radiation plants are usually set to deliver a particular dosage over a set period of time. Although the time can be varied to vary dosage the normal procedure is to adapt the dosage to the requirements of the largest entity being sterilized and simply over-dose smaller products. Of course, if all products are the same, this is not an issue. The sterilization time is generally in the 4 to 8 hour range and the possibility of product degradation in the form of discoloration and embrittlement is greater than with other forms of radiation sterilization such as electron beam sterilization.

Electron beam or e-beam sterilization is similar to gamma sterilization in that it involves the generation of ionizing energy that on contact with a product to be sterilized interacts with electrons comprising chemical bonds, including those in the reproductive cells of microorganisms, altering the bonds and molecules, such as DNA, RNA and the like, thereby killing the microorganisms or at least destroying their ability to reproduce. E-beam sterilization, however, is safer, faster and substantially more flexible that gamma radiation. It is safer because it does not involve the use of radioactive isotopes. Rather, the energy is created by the acceleration and conversion of electricity. It is more flexible in that, while many current commercial e-beam accelerators operate at a single energy, usually from about 3 MeV (million electron volts) to about 12 MeV, equipment capable of operating at various energies is available. E-beam is faster that gamma irradiation in that products remain in an electron beam cell for a matter of seconds compared to several minutes or hours in a gamma cell. Since exposure time is less, degradation of the product being sterilized is also reduced. Unfortunately, with regard to drug-carrying implantable medical devices such as drug-eluting stents, degradation of often hypersensitive biologically active molecules remains a problem. For instance, in a drug stability test involving poly(d,l-lactide) polymer containing everolimus coated on a stent, it was found that 3 to 5% of the drug was lost due to degradation as the result of e-beam sterilization.

What is needed is a method of protecting drugs that are coated on implantable medical devices from the effects of e-beam sterilization while not deleteriously affecting the overall sterilization process. The current invention provides such a method and devices based thereon.

### SUMMARY

Thus, an aspect of this invention is an implantable medical device, comprising:

a device body;
an optional primer layer disposed over the device body;
a drug reservoir layer disposed over the device body or over the primer layer, if opted; the drug reservoir layer comprising one or more therapeutic agents;
an optional rate-controlling layer disposed over the drug reservoir layer;
an optional topcoat layer disposed over the drug reservoir layer or the rate-controlling layer, if opted; and,
a stable nitroxide, wherein:
the stable nitroxide is contained in the drug reservoir layer, in the rate-controlling layer, in the topcoat layer, in a separate layer disposed between the drug reservoir layer and the external environment, or in any combination of these.

In an aspect of this invention, the stable nitroxide comprises a polymer-bound stable nitroxide.

In an aspect of this invention wherein the polymer-bound stable nitroxide comprises a covalent bond between the stable nitroxide and the polymer.

In an aspect of this invention, the polymer is a poly(ester-amide).

In an aspect of this invention, the poly(ester-amide) has the formula:

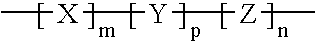

wherein:
m is a number from 0 to 1, inclusive;
p is a number from 0 to 1, inclusive;
n is a number from 0 to 1, inclusive, wherein:

m+p+n=1;

X has the chemical structure:

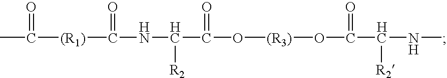

Y has the chemical structure:

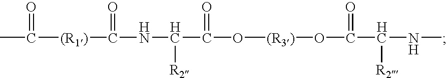

Z has the chemical structure:

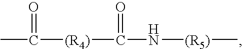

wherein:
R₁, R_{1′} and R₄ are independently selected from the group consisting of (1C-12C)alkyl and (2C-12C)alkenyl, with the proviso that if R₃ and R_{3′} are the same, then R₁ and R_{1′} are different;
R₂, R_{2′}, R_{2″} and R_{2′″} are independently selected from the group consisting of hydrogen and (1C-4C)alkyl, wherein:
   the alkyl group is optionally substituted with a moiety selected from the group consisting of —OH, —O(1C-4C)alkyl, —SH, —S(1C-4C)alkyl, —SeH, —COR₆, —NHC(NH)NH₂, imidazol-2-yl, imidazole-5-yl, indol-3-yl, phenyl, 4-hydroxyphenyl and 4-[(1C-4C)alkylO]phenyl, wherein:
      R₆ is selected from the group consisting of —OH, —O(1C-4C)alkyl, —NH₂, —NH(1C-4C)alkyl,
      —N(1C-4C)alkyl(1C-4C)alkyl₂, a stable nitroxide, and

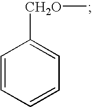

or
one or more of R₂, R_{2′}, R_{2″} and R₂′″ may form a bridge between the carbon to which it is attached and the adjacent nitrogen, the bridge comprising —CH₂CH₂CH₂—;
R₃ and R_{3′} are independently selected from the group consisting of (1C-12C)alkyl, (2C-12C)alkenyl, (3C-8C)cycloalkyl and —(CH₂CH₂O)_{q}CH₂CH₂—, wherein q is an integer from 1 to 10, inclusive, with the proviso that if R₁ and R_{1′} are the same then R₃ and R_{3′} are different; and,
R₅ is selected from the group consisting of —CH(COR₆)CH₂S—,

—CH(COR₆)CH₂O—, —CH(COR₆)(CH₂)₄NH—, —(CH₂)₄CH(COR₆)NH—,

—CH(COR₆)CH(CH₃)O—,

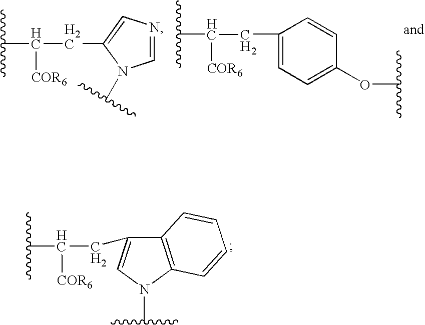

wherein at least one of R₂, R_{2′}, R_{2″}, R_{2′″} and R₅ comprises R₆, wherein R₆ comprises a stable nitroxide.

In an aspect of this invention, R₁, R_{1′} and R₄ are independently selected from the group consisting of —(CH₂)₄— and —(CH₂)₈—.

In an aspect of this invention, R₂, R_{2′}, R_{2″} and R_{2′″} are independently selected from the group consisting of —CH₃, —H₂CH₂NHC(NH)NH₂, —CH₂CONH₂,

### —CH₂COOH, —CH₂SH, —CH₂CH₂COOH, —CH₂CH₂CONH₂, —CH₂NH₂,

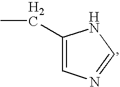

### —CH(CH₃)CH₂CH₃.—CH₂CH(CH₃)₂, —(CH₂)₄NH₂, (CH₂)₂SCH₃,

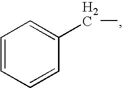

### CH₂OH,

### —CH(CH₃)OH,

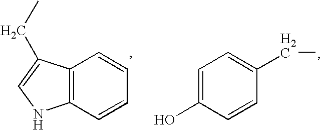

### CH(CH₃)₂ and —CH₂CH₂CH₂—, wherein the second carbon is covalently bonded to the nitrogen adjacent to the carbon to which R₂ is bonded.

In an aspect of this invention, R₂, R_{2′, R}_{2″} and R_{2′″} are —CH₂CH(CH₃)₂.

In an aspect of this invention, R₃ is —(CH₂)₆— and R_{3′} is

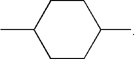

In an aspect of this invention, R₅ is —(CH₂)₄COR₆NH— and R₆ comprises a stable nitroxide.

In an aspect of this invention, the stable nitroxide is selected from the group consisting of:

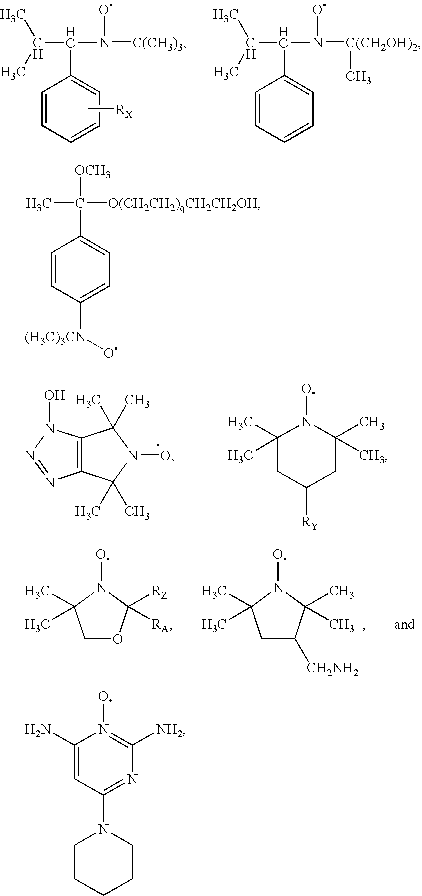

wherein Rₓ and R_{y} are selected from the group consisting of —NH₂, —OH and C(O)OH; R_{Z} is —(CH₂)_{b}C(O)OH and R_{A} is —(CH₂)_{b′}CH₃, where b and b′ are independently 1-16.

In an aspect of this invention, the stable nitroxide is

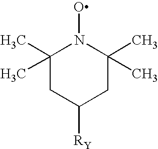

wherein R_{y} is —NH₂.

In an aspect of this invention, p=0.

In an aspect of this invention, when p=0, R₁ and R₄ are independently selected from the group consisting of —(CH₂)₄— and —(CH₂)₈—.

In an aspect of this invention, when p=0, R₂ and R₂′ are independently selected from the group consisting of —CH₃, —CH₂CH₂NHC(NH)NH₂, —CH₂CONH₂, —CH₂COOH, —CH₂SH, —CH₂CH₂COOH, —CH₂CH₂CONH₂, —CH₂NH₂,

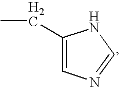

### —CH(CH₃)CH₂CH₃.—CH₂CH(CH₃)₂, —(CH₂)₄NH₂, (CH₂)₂SCH₃,

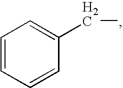

### CH₂OH, —CH(CH₃)OH,

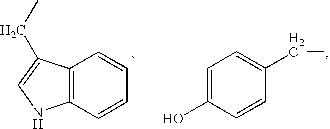

### CH(CH₃)₂ and —CH₂CH₂CH₂—, wherein the second carbon is covalently bonded to the nitrogen adjacent to the carbon to which R₂ is bonded.

In an aspect of this invention, when p=0, R₂ and R_{2′}, are —CH₂CH(CH₃)₂.

In an aspect of this invention, when p=0, R₃ is selected from the group consisting of —(CH₂)₃—, —(CH₂)₆— and —(CH₂CH₂O)_{q}CH₂CH₂—, wherein q is an integer from 1 to 10, inclusive.

In an aspect of this invention, when p=0, q is 2.

In an aspect of this invention, when p=0, R₅ is —(CH₂)₄CH(COR₆)NH— and R₆ comprises a stable nitroxide.

In an aspect of this invention, when p=0, the stable nitroxide is selected from the group consisting of:

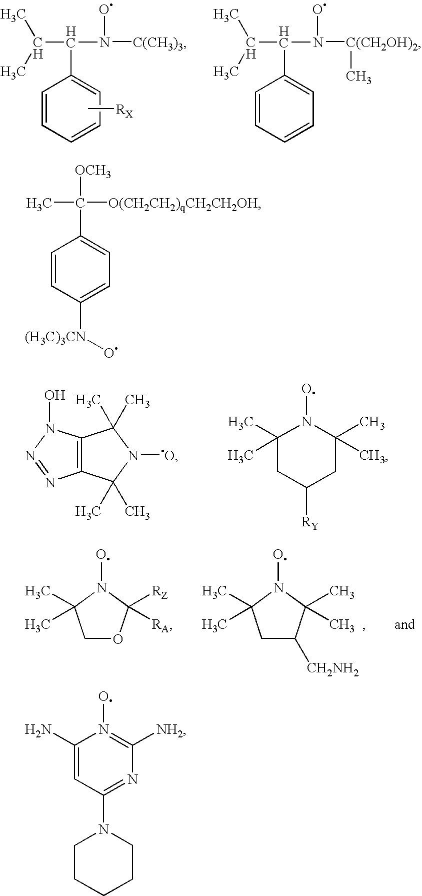

wherein Rₓ and R_{y} are selected from the group consisting of —NH₂, —OH and C(O)OH; R_{Z} is —(CH₂)_{b}C(O)OH and R_{A} is —(CH₂)_{b′}CH₃, where b and b′ are independently 1-16.

In an aspect of this invention, when p=0, the stable nitroxide is

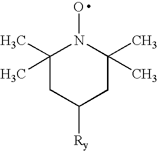

wherein R_{y} is —NH₂.

In an aspect of this invention, p and n are 0.

In an aspect of this invention, when p and n are 0, R₁ is selected from the group consisting of —(CH₂)₄— and —(CH₂)₈—.

In an aspect of this invention, when p and n are 0, R₂ and R_{2′} are independently selected from the group consisting of —CH₃, —H₂CH₂NHC(NH)NH₂, —CH₂CONH₂, —CH₂COOH, —CH₂SH, —CH₂CH₂COOH, —CH₂CH₂CONH₂, —CH₂NH₂,

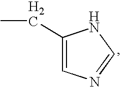

### —CH(CH₃)CH₂CH₃.—CH₂CH(CH₃)₂, —(CH₂)₄NH₂, (CH₂)₂SCH₃,

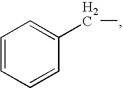

### CH₂OH, —CH(CH₃)OH,

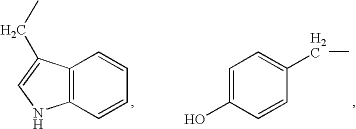

### CH(CH₃)₂ and —CH₂CH₂CH₂—, wherein the second carbon is covalently bonded to the nitrogen adjacent to the carbon to which R₂ is bonded.

In an aspect of this invention, when p and n are 0, R₂ and R_{2′} are CH₂CH(CH₃)₂.

In an aspect of this invention, when p and n are 0, R₃ is selected from the group consisting of —(CH₂)₃—, —(CH₂)₆— and —(CH₂CH₂O)_{q}CH₂CH₂—, wherein q is an integer from 1 to 10, inclusive.

In an aspect of this invention, when p and n are 0, q is 2.

In an aspect of this invention, when p and n are 0, R₅ is —(CH₂)₄CH(COR₆)NH—, wherein R₆ comprises a stable nitroxide.

In an aspect of this invention, when p and n are 0, the stable nitroxide is selected from the group consisting of:

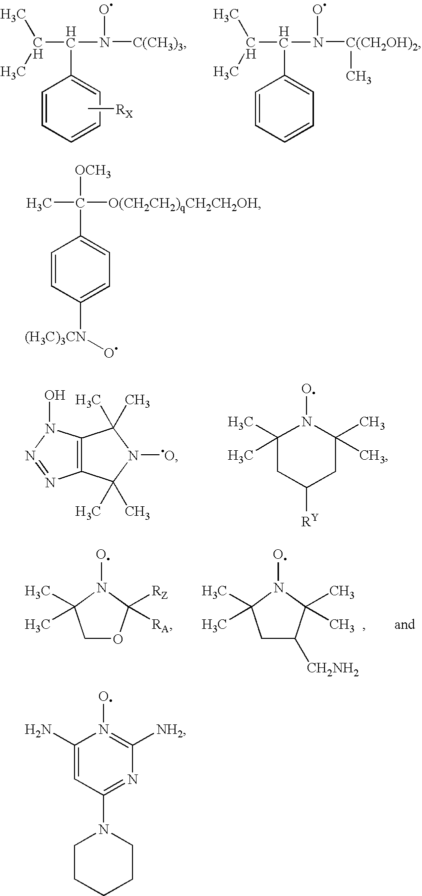

wherein Rₓ and R_{y} are selected from the group consisting of —NH₂, —OH and C(O)OH; R_{Z} is —(CH₂)_{b}C(O)OH and R_{A} is —(CH₂)_{b′}CH₃, where b and b′ are independently 1-16.

In an aspect of this invention, when p and n are 0, the stable nitroxide is

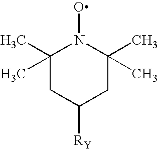

wherein R_{y} is —NH₂.

In an aspect of this invention, the therapeutic agent is everolimus.

In an aspect of this invention, the implantable medical device is a stent.

An aspect of this invention is a method comprising:
providing an implantable medical device, wherein the device comprises:
   a device body;
   an optional primer layer disposed over the device body;
   a drug reservoir layer disposed over the device body or over the primer layer, if opted; the drug reservoir layer comprising one or more therapeutic agents;
   an optional rate-controlling layer disposed over the drug reservoir layer;
   an optional topcoat layer disposed over the drug reservoir layer or the rate-controlling layer, if opted; and,
   a stable nitroxide, wherein:
      the stable nitroxide is contained in the drug reservoir layer, in the rate-controlling layer, in the topcoat layer, in a separate layer disposed between the drug reservoir layer and the external environment, or in any combination of these; and,
         sterilizing the device using a free-radical generating sterilization method.

In an aspect of this invention, in the method herein, the free-radical generating sterilization method is e-beam sterilization.

In an aspect of this invention, in the method herein, the stable nitroxide is a polymer-bound stable nitroxide.

In an aspect of this invention, in the method herein, the polymer is a poly(ester-amide).

In an aspect of this invention, in the method herein, the poly(ester-amide) has the formula:

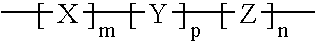

wherein:
m is a number from 0 to 1, inclusive;
p is a number from 0 to 1, inclusive;
n is a number from 0 to 1, inclusive, where:

m+p+n=1;

X has the chemical structure:

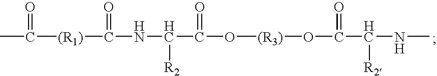

Y has the chemical structure:

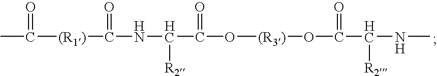

Z has the chemical structure:

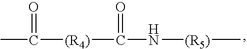

wherein:
R₁, R_{1′} and R₄ are independently selected from the group consisting of (1C-12C)alkyl and (2C-12C)alkenyl, with the proviso that if R₃ and R_{3′} are the same, then R₁ and R_{1′} are different;
R₂, R_{2′}, R_{2″} and R_{2′″} are independently selected from the group consisting of hydrogen and (1C-4C)alkyl, wherein:
   the alkyl group is optionally substituted with a moiety selected from the group consisting of —OH, —O(1C-4C)alkyl, —SH, —S(1C-4C)alkyl, —SeH, —COR₆, —NHC(NH)NH₂, imidazol-2-yl, imidazole-5-yl, indol-3-yl, phenyl, 4-hydroxyphenyl and 4-[(1C-4C)alkylO]phenyl, wherein:
      R₆ is selected from the group consisting of —OH, —O(1C-4C)alkyl, —NH₂, —NH(1C-4C)alkyl,
      —N(1C-4C)alkyl₁(1C-4C)alkyl₂, a stable nitroxide, and

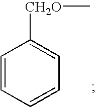

or
one or more of R₂, R_{2′}, R_{2″} and R_{2′″} may form a bridge between the carbon to which it is attached and the adjacent nitrogen, the bridge comprising —CH₂CH₂CH₂—;
R₃ and R₃ are independently selected from the group consisting of (1C-12C)alkyl, (2C-12C)alkenyl, (3C-8C)cycloalkyl and —(CH₂CH₂O)_{q}CH₂CH₂—, wherein q is an integer from 1 to 10, inclusive, with the proviso that if R₁ and R_{1′} are the same then R₃ and R_{3′} are different; and,
R₅ is selected from the group consisting of —CH(COR₆)CH₂S—, —CH(COR₆)CH₂O—, —CH(COR₆)(CH₂)₄NH—, —(CH₂)₄CH(COR₆)NH—, —CH(COR₆)CH(CH₃)O—,

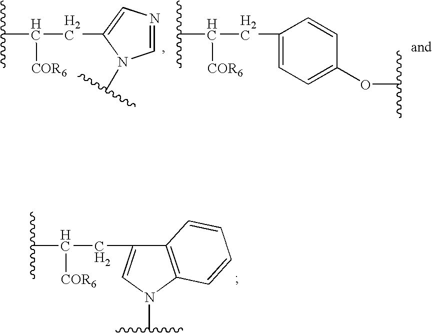

wherein at least one of R₂, R_{2′}, R_{2″}, R_{2′″} and R₅ comprises R₆, wherein R₆ comprises a stable nitroxide.

In an aspect of this invention, in the method herein, the stable nitroxide is selected from the group consisting of:

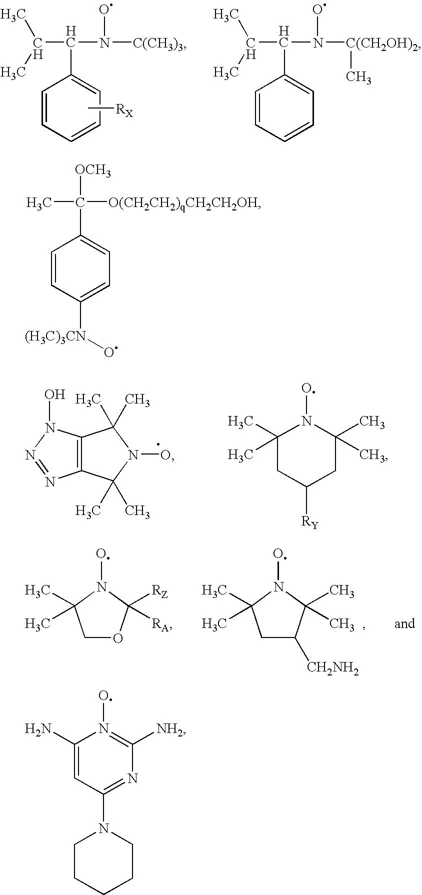

wherein Rₓ and R_{y} are selected from the group consisting of —NH₂, —OH and C(O)OH; R_{Z} is —(CH₂)_{b}C(O)OH and R_{A} is —(CH₂)_{b}CH₃, where b and b′ are independently 1-16.

In an aspect of this invention, in the method herein, the stable nitroxide is

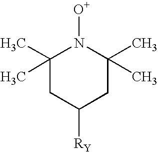

wherein R_{y} is —NH₂.

In an aspect of this invention, in the method herein, the implantable medical device is a stent.

In an aspect of this invention, in the method herein, the therapeutic agent is everolimus.

### DETAILED DESCRIPTION

The current invention provides a method of protecting therapeutic agents coated on implantable medical devices such as drug-eluting stents from the effects of sterilization procedures involving radiolytically-generated free radicals such as e-beam sterilization by providing free radical scavengers in the form of stable paramagnetic nitroxides (referred to hereafter simply as “stable nitroxides”). While being readily available to quench free radicals formed on drug molecules adhered to the device, they would not be available to quench free radicals formed within the cell structure of microorganisms and therefore would not be expected to interfere with the sterilization process.

Use of the singular herein includes the plural and visa versa unless expressly stated to be otherwise. That is, “a” and “the” refer to one or more of whatever the word modifies. For example, “a therapeutic agent” includes one such agent, two such agents, etc. Likewise, “the layer” may refer to one, two or more layers and “the polymer” may mean one polymer or a plurality of polymers. By the same token, words such as, without limitation, “layers” and “polymers” would refer to one layer or polymer as well as to a plurality of layers or polymers unless, again, it is expressly stated or obvious from the context that such is not intended.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves and cerebrospinal fluid shunts.

An implantable medical device specifically designed and intended solely for the localized delivery of a therapeutic agent is within the scope of this invention.

As used herein, “device body” refers to a fully-formed usable implantable medical device with an outer surface to which no coating or layer of material different from that of which the device itself is manufactured has been applied. By “outer surface” is meant any surface however spatially oriented that is in contact with bodily tissue or fluids. A common example of a “device body” is a BMS, i.e., a bare metal stent, which, as the name implies, is a fully-formed usable stent that has not been coated with a layer of any material different from the metal of which it is made on any surface that is in contact with bodily tissue or fluids. Of course, device body refers not only to BMSs but to any uncoated device regardless of what it is made of.

Implantable medical devices made of virtually any material, i.e., materials presently known to be useful for the manufacture of implantable medical devices and materials that may be found to be so in the future, may be used with a coating of this invention. For example, without limitation, an implantable medical device useful with this invention may be made of one or more biocompatible metals or alloys thereof including, but not limited to, cobalt-chromium alloy (ELGILOY, L-605), cobalt-nickel alloy (MP-35N), 316L stainless steel, high nitrogen stainless steel, e.g., BIODUR 108, nickel-titanium alloy (NITINOL), tantalum, platinum, platinum-iridium alloy, gold and combinations thereof.

Implantable medical devices may also be made of polymers that are biocompatible and biostable or biodegradable, the latter term including bioabsorbable and/or bioerodable.

As used herein, “biocompatible” refers to a polymer that both in its intact, that is, as synthesized, state and in its decomposed state, i.e., its degradation products, is not, or at least is minimally, toxic to living tissue; does not, or at least minimally and reparably, injure(s) living tissue; and/or does not, or at least minimally and/or controllably, cause(s) an immunological reaction in living tissue.

Among useful biocompatible, relatively biostable polymers are, without limitation polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins.

Biocompatible, biodegradable polymers include naturally-occurring polymers such as, without limitation, collagen, chitosan, alginate, fibrin, fibrinogen, cellulosics, starches, dextran, dextrin, hyaluronic acid, heparin, glycosaminoglycans, polysaccharides and elastin.

One or more synthetic or semi-synthetic biocompatible, biodegradable polymers may also be used to fabricate an implantable medical device useful with this invention. As used herein, a synthetic polymer refers to one that is created wholly in the laboratory while a semi-synthetic polymer refers to a naturally-occurring polymer than has been chemically modified in the laboratory. Examples of synthetic polymers include, without limitation, polyphosphazines, polyphosphoesters, polyphosphoester urethane, polyhydroxyacids, polyhydroxyal kanoates, polyan hydrides, polyesters, polyorthoesters, polyamino acids, polyoxymethylenes, poly(ester-amides) and polyimides.

Blends and copolymers of the above polymers may also be used and are within the scope of this invention. Based on the disclosures herein, those skilled in the art will recognize those implantable medical devices and those materials from which they may be fabricated that will be useful with the coatings of this invention. At present, preferred implantable medical devices for use with the coatings of this invention are stents.

A stent refers generally to any device used to hold tissue in place in a patient's body. Particularly useful stents, however, are those used for the maintenance of the patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, restenosis and vulnerable plaque. Vulnerable plaque (VP) refers to a fatty build-up in an artery thought to be caused by inflammation. The VP is covered by a thin fibrous cap that can rupture leading to blood clot formation. A stent can be used to strengthen the wall of the vessel in the vicinity of the VP and act as a shield against such rupture. A stent can be used in, without limitation, neuro, carotid, coronary, pulmonary, aorta, renal, biliary, iliac, femoral and popliteal as well as other peripheral vasculatures. A stent can be used in the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

In addition to the above uses, stents may also be employed for the localized delivery of therapeutic agents to specific treatment sites in a patient's body. In fact, therapeutic agent delivery may be the sole purpose of the stent or the stent may be primarily intended for another use such as those discussed above with drug delivery providing an ancillary benefit.

A stent used for patency maintenance is usually delivered to the target site in a compressed state and then expanded to fit the vessel into which it has been inserted. Once at a target location, a stent may be self-expandable or balloon expandable. In any event, due to the expansion of the stent, any coating thereon must be flexible and capable of elongation.

As used herein, “optional” means that the element modified by the term may or may not be present. For example, without limitation, a device body (db) that has coated on it an “optional” primer layer (pl), a drug reservoir layer (dr), an “optional” rate-controlling layer (rc), an “optional” top-coat layer (tc) and a stable nitroxide layer (sn) refers, without limitation, to any of the following devices: db+dr+sn, db+pl+dr+sn, db+dr+rc+sn, db+pl+dr+rc+sn, db+dr+tc+sn, db+pl+dr+tc+sn, db+dr+rc+tc+sn or db+pl+dr+rc+tc+sn.

As used herein, a “primer layer” refers to a coating consisting of a polymer or blend of polymers that exhibit good adhesion characteristics with regard to the material of which the device body is manufactured and good adhesion characteristic with regard to whatever material is to be coated on the device body. Thus, a primer layer seves as an adhesive intermediary layer between a device body and materials to be carried by the device body and is, therefore, applied directly to the device body. Examples without limitation, of primers include acrylate and methacrylate polymers with poly(n-butyl methacrylate) being a presently preferred primer.

As use herein, a material that is described as a layer “disposed over” an indicated substrate, e.g., without limitation, a device body or another layer, refers to a relatively thin coating of a material applied, preferably at present, directly to essentially the entire exposed surface of the indicated substrate. By “exposed surface” is meant that surface of the substrate that, in use, would be in contact with bodily tissues or fluids. “Disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, “drug reservoir layer” refers either to a layer of one or more therapeutic agents applied neat or to a layer of polymer or blend of polymers that has dispersed within its three-dimensional structure one or more therapeutic agents. A polymeric drug reservoir layer is designed such that, by one mechanism or another, e.g., without limitation, by elution or as the result of biodegradation of the polymer, the therapeutic substance is released from the layer into the surrounding environment.

As used herein, “therapeutic agent” refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. As used herein, a therapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

As used herein, the terms “drug” and “therapeutic agent” are used interchangeably.

As used herein, “rate-controlling layer” refers to a polymeric layer that is applied over a drug reservoir layer to modify the rate of release into the environment of the therapeutic agents from the drug reservoir layer. A rate-controlling layer may be used simply to “tune” the rate of release of a therapeutic agent to exactly that desired by the practitioner or it may be a necessary adjunct to the construct because the polymer or blend of polymers with which the therapeutic agent is compatible with regard to coating as a drug reservoir layer may be too permeable to the therapeutic substance resulting in too rapid release and delivery of the therapeutic substance into a patient's body. A non-limiting example is an everolimus drug reservoir layer comprising PEA-TEMPO (a poly(ester-amide) to which 2,2,6,6-tetramethyl-4-aminopiperidine-1-oxyl has been covalently appended). While PEA-TEMPO has very desirable in vivo properties, it is quite permeable to everolimus. Thus, sustained release (i.e., release of a therapeutically effective amount of a drug over an extended period of time which may be a few days, a few months or more) of everolimus from a poly(ester-amide) polymer matrix is difficult and in some cases impossible to achieve. To ameliorate this situation, a rate-controlling polymer or blend of polymers through which everolimus is more controllably permeable can be applied over the PEA-TEMPO layer. The reduced permeability of everolimus through the rate-controlling layer may be due, without limitation, to inherent characteristics of the polymer and the way it interacts with everolimus (or any other therapeutic agent) or it may be due to such factors as cross-linking of the rate-controlling polymer, etc.

As used herein, a “topcoat layer” refers to an outermost layer, that is, a layer that is in contact with the external environment and that is coated over all other layers. The topcoat layer may be applied to provide better hydrophilicity to the device, to better lubricate the device or merely as a physical protectant of the underlying layers. The topcoat layer, however, may also contain therapeutic agents, in particular if the treatment protocol being employed calls for essentially immediate release of one or more therapeutic agent (these being included in the topcoat layer) followed by the controlled release of another therapeutic agent or agents over a longer period of time. In addition, the topcoat layer may contain one or more “biobeneficial agents.”

A “biobeneficial” agent is one that beneficially affects an implantable medical device by, for example, reducing the tendency of the device to protein foul, increasing the hemocompatibility of the device, and/or enhancing the non-thrombogenic, non-inflammatory, non-cytotoxic, non-hemolytic, etc. characteristics of the device. Some representative biobeneficial materials include, but are not limited to, polyethers such as poly(ethylene glycol) (PEG) and poly(propylene glycol); copoly(ether-esters) such as poly(ethylene oxide-co-lactic acid); polyalkylene oxides such as poly(ethylene oxide) and poly(propylene oxide); polyphosphazenes, phosphoryl choline, choline, polymers and copolymers of hydroxyl bearing monomers such as hydroxyethyl methacrylate, hydroxypropyl methacrylate, hydroxypropylmethacrylamide, poly(ethylene glycol)acrylate, 2-methacryloyloxyethylphosphorylcholine (MPC) and n-vinyl pyrrolidone (VP); carboxylic acid bearing monomers such as methacrylic acid, acrylic acid, alkoxymethacrylate, alkoxyacrylate, and 3-trimethylsilylpropyl methacrylate; polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functionalized poly(vinyl pyrrolidone); biomolecules such as fibrin, fibrinogen, cellulose, starch, collagen, dextran, dextrin, hyaluronic acid, heparin, glycosamino glycan, polysaccharides, elastin, chitosan, alginate, silicones, PolyActive™, and combinations thereof. PolyActive™ refers to a block copolymer of poly(ethylene glycol) and poly(butylene terephthalate).

As used herein, a “stable nitroxide” refers to an isolatable paramagnetic organic compound having the generic structure RR′N—O. wherein R and R′ may be aliphatic, aromatic or heterocyclic, where the heterocycle is aromatic or non-aromatic, or R and R′ may join together to form a ring which may be acyclic or aromatic. Some exemplary stable nitroxides include, without limitation:

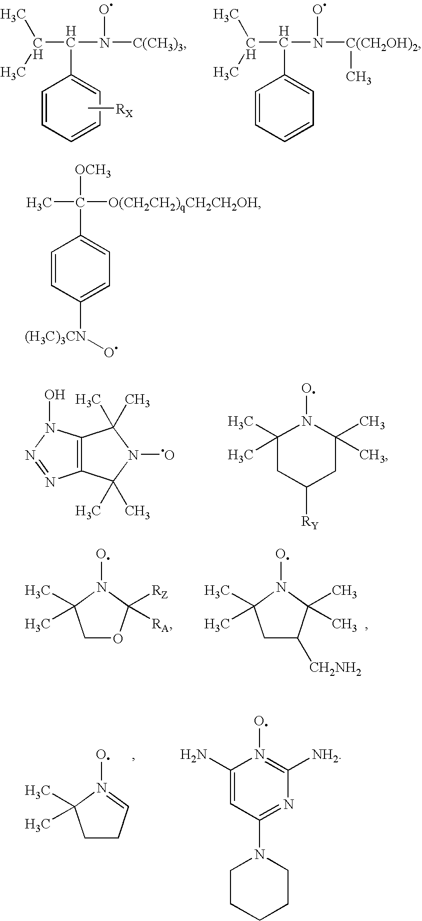

In these non-limiting examples, Rₓ and R_{y} may be such groups as —NH₂, —OH, —COOH, etc., R_{z} may be, without limitation, —(CH₂)_{b}C(O)OH and R_{A} may be, again without limitation, —(CH₂)_{b′}CH₃, where b and b′ are independently 1-16. For the purposes of this invention, it is presently preferred that the stable nitroxide be covalently bonded to a polymer. Therefore, the stable nitroxide must contain at least one functional group that can react with a functional group on the polymer. Since poly(ester-amides) are presently preferred as polymers for use herein and it is further presently preferred that the amide-forming entity be one of the 20 essential amino acids plus selenoadenine, which amino acids include —OC(O)OH, —OH, —SH and —NH₂ groups, the function group on the stable nitroxide should be one that can react with one or more of these groups. For example, without limitation, 4-amino-2,2,6,6-tetramethylpiperidine-1-oxyl can be reacted with a pendant carboxylic acid group of lysine that has been included in one of the constitutional units of the polymer backbone, to form an amide. Other such functional groups that will afford covalently bonded stable nitroxides will be evident to those skilled in the art based on the disclosures herein and are within the scope of this invention. Some of the representative stable nitroxides above are shown with functional groups appropriate for use herein.

A poly(ester-amide) refers to a polymer that has in its backbone structure both ester and amide bonds. The poly(ester-amides) of this invention have the generic formula:

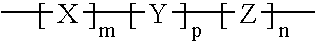

wherein X, Y and Z refer to the constitutional units of the polymer. The numbers m, p and n refer to decimal fractions between 0 and 1, inclusive of 1 and 0, and describe the mole fraction of each constitutional unit in the polymer. For example without limitation, m=0.5, p=0.25 and n=0.25 would mean that the poly(ester-amide) is comprised of 50 mol % X, 25 mol % Y and 25 mol % Z. This means, of course, that m+p+n must equal 1.0. If any of m, p or n is 0, it simply means that that constitutional unit is missing from the polymer. For example, without limitation, if p=0, the resulting polymer would have the generic structure:

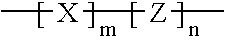

and m+n would equal 1.0.

Presently preferred poly(ester-amides) are either tri-block copolymers of X, Y and Z, di-block copolymers of X and Z or homopolymers of constitutional unit X. In general, the constitutional units X and Y comprise an amino acid that is reacted with a diol to give a diamino ester, which is then reacted with a diacid. A non-limiting example would be the reaction of 1,6-hexane diol with 1-leucine to give the diamino diester, which is then reacted with sebacic acid to provide X or Y.

While any amino acid may be used to construct a poly(ester-amide) of this invention, particularly useful amino acids are the so-called essential amino acids of which there currently 20: alanine, arginine, asparagine, aspartic acid, cysteine, glutamic acid, glutamine, glycine, histidine, isoleucine, leucine, lysine, methionine, phenyl alanine, proline, serine, threonine, tryptophan, tyrosine and valine. More recently selenoadenine has been found to be incorporated into a number of naturally-occurring proteins and is included as a particularly useful amino acid of this invention. In naturally-occurring biological proteins, these amino acids appear as the 1-enantiomeric isomers but for the purposes of this invention they may be used as their I- or d-enantiomers or as racemic mixtures.

Constitutional unit Z, on the other hand, is the result of the reaction of a diacid with a tri-functional amino acid wherein two of the functional groups are capable of reacting with the diacid. As example would be the reaction of sebacic acid or an activated derivative thereof, with 1-lysine, 2,6-diaminohexanoic acid.

As used herein, “alkyl” refers to a straight or branched chain fully saturated (no double or triple bonds) hydrocarbon (carbon and hydrogen only) group. The alkyl groups of this invention may range from C₁ to C₁₂, preferably C₂ to C₁₀ and currently most preferably C₃ to C₈. Examples of alkyl groups include, but are not limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, and cyclohexyl.

As used herein, “mC to nC,” wherein m and n are integers refers to the number of possible carbon atoms in the indicated group. That is, the group can contain from “m” to “n”, inclusive, carbon atoms. An alkyl group of this invention may comprise from 1 to 12 carbon atoms, that is m may be 1 and n may be 12. Of course, a particular alkyl group may be more limited, for instance without limitation, to 3 to 8 carbon atoms, in which case it would be designate as a (3C-8C)alkyl group. The numbers are inclusive and incorporate all straight or branched chain structures having the indicated number of carbon atoms. For example without limitation, a “C₁ to C₄ alkyl” group refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)— and (CH₃)₃CH—.

As use herein, “cycloalkyl” refers to an alkyl group in which the end carbon atoms of the alkyl chain are covalently bonded to one another. The numbers “m” to “n” then refer to the number of carbon atoms in the ring so formed. Thus for instance, a (3C-8C)cycloalkyl group refers to a three, four, five, six, seven or eight member ring, that is, cyclopropane, cyclobutane, cyclopentane, cyclohexane, cycloheptane and cyclooctane.

As used herein, “alkenyl” refers to an alkyl group that contains in the straight or branched hydrocarbon chain one or more double bonds.

Whenever a group of this invention is described as being “optionally substituted” that group may be unsubstituted or substituted with one or more of the indicated substituents.

Standard shorthand designations well-known to those skilled in the art are used throughout this application. Thus the intended structure will easily be recognizable to those skilled in the art based on the required valency of any particular atom with the understanding that all necessary hydrogen atoms are provided. For example, —COR, because carbon is tetravalent, must refer to the structure

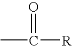

as that is the only way the carbon can be tetravalent without the addition of unshown hydrogen or other atoms.

An implantable medical device of this invention includes one or more therapeutic agents. Virtually any therapeutic agent found to be useful when incorporated on and implantable medical device may be used in the device and method of this invention. Examples of therapeutic agents include, but are not limited to anti-proliferative, anti-inflammmatory, antineoplastic, antiplatelet, anti-coagulant, anti-fibrin, antithrombonic, antimitotic, antibiotic, antiallergic and antioxidant compounds. Thus, the therapeutic agent may be, again without limitation, a synthetic inorganic or organic compound, a protein, a peptide, a polysaccharides and other sugars, a lipid, DNA and RNA nucleic acid sequences, an antisense oligonucleotide, an antibodies, a receptor ligands, an enzyme, an adhesion peptide, a blood clot agent such as streptokinase and tissue plasminogen activator, an antigen, a hormone, a growth factor, a ribozyme, a retroviral vector, an anti-proliferative agent such as rapamycin, 40-O—(2-hydroxy)ethyl-rapamycin (everolimus), paclitaxel, docetaxel, methotrexate, azathioprine, vincristine, vinblastine, fluorouracil, doxorubicin hydrochloride, and mitomycin, an antiplatelet compound, an anticoagulant, an antifibrin, an antithrombins such as sodium heparin, a low molecular weight heparin, a heparinoid, hirudin, argatroban, forskolin, vapiprost, prostacyclin, a prostacyclin analogue, dextran, D-phe-pro-arg-chloromethylketone (synthetic antithrombin), dipyridamole, glycoprotein IIb/IIIa platelet membrane receptor antagonist antibody, recombinant hirudin, a thrombin inhibitor such as Angiomax ä, a calcium channel blocker such as nifedipine, colchicine, a fibroblast growth factor (FGF) antagonist, fish oil (omega 3-fatty acid), a histamine antagonist, lovastatin, a monoclonal antibodie, nitroprusside, a phosphodiesterase inhibitor, a prostaglandin inhibitor, suramin, a serotonin blocker, a steroid, a thioprotease inhibitor, triazolopyrimidine, a nitric oxide or nitric oxide donor, a super oxide dismutase, a super oxide dismutase mimetic, estradiol, an anticancer agent, a dietary supplement such as vitamins, an anti-inflammatory agent such as aspirin, tacrolimus, dexamethasone and clobetasol, a cytostatic substance such as angiopeptin, an angiotensin converting enzyme inhibitor such as captopril, cilazapril or lisinopril, an antiallergic agent such as permirolast potassium, alpha-interferon, bioactive RGD, and genetically engineered epithelial cells. Other therapeutic agents which are currently available or that may be developed in the future for use with implantable medical devices may likewise be used and all are within the scope of this invention.

With regard to the present invention, everolimus, an immunosuppressive macrolide antibiotic, is a presently preferred therapeutic agent.

To be effective, the stable nitroxide of this invention either be included in the same layer as the therapeutic agent, i.e., the drug reservoir layer, or in any layer, including its own separate layer disposed between the drug reservoir layer and free radial source, for example without limitation, an e-beam generator.

### EXAMPLES

### Example 1

This example illustrates how one of the polymer-bound stable nitroxides of this invention can be used to protect a therapeutic agent, in this example everolimus, from the effects of e-beam sterilization.

A drug reservoir layer containing everolimus and a poly(ester-amide) with either 4-amino-2,2,6,6-tetramethylpiperidine-1-oxyl, a stable nitroxide known as 4-aminoTEMPO, covalently bonded to a lysine carboxyl group as one of the amino acids in the polymer backbone or with a benzyl group covalently bonded to the same carboxyl group was spray coated on small 12 mm Vision® stents in such a manner that 392 μg of total dry weight material were deposited, which corresponds to a drug loading of 100 μg/cm².

A topcoat consisting of the same polymer-bound stable nitroxide or the benzyl group was applied so as to give a total dry weight of material in the topcoat of about 400 μg. Each layer was applied as a 2 wt % solution in absolute ethanol. The stents were then subjected to e-beam sterilization.

Table 1 shows the drug purity and recovery before and after e-beam sterilization (for each test n=6). While the protection afforded by the stable nitroxide is not obvious from the purity comparison, it is readily apparent from the recovery percentage. Thus, the stable nitroxide-containing samples lost about 2.5% of the everolimus as the result of sterilization, all other factors being held equal, while the benzyl-containing samples lost about 5.6%.

TABLE 1

| | Non-sterile | Non-sterile | | Sterile |
|---|---|---|---|---|
| Polymer | Purity | % Recovery | Sterile Purity | % Recovery |
| PEA-TEMPO | 100 ± 0 | 94.4 ± 0.2 | 99.8 ± 0.1 | 92.0 ± 1.4 |
| PEA-BZ | 99.7 ± 0.0 | 91.9 ± 0.5 | 99.5 ± 0.0 | 86.8 ± 0.6 |

What is claimed:

1. An implantable medical device, comprising:
a device body;
an optional primer layer disposed over the device body;
a drug reservoir layer disposed over the device body or over the primer layer, if opted; the drug reservoir layer comprising one or more therapeutic agents;
an optional rate-controlling layer disposed over the drug reservoir layer;
an optional topcoat layer disposed over the drug reservoir layer or the rate-controlling layer, if opted; and,
a stable nitroxide, wherein:
the stable nitroxide is contained in the drug reservoir layer, in the rate-controlling layer, in the topcoat layer, in a separate layer disposed between the drug reservoir layer and the external environment, or in any combination of these.

2. The implantable medical device of claim 1, wherein the stable nitroxide comprises a polymer-bound stable nitroxide.

3. The implantable medical device of claim 2, wherein the polymer-bound stable nitroxide comprises a covalent bond between the stable nitroxide and the polymer.

4. The implantable medical device of claim 3, wherein the polymer is a poly(ester-amide).

5. The implantable medical device of claim 4, wherein the poly(ester-amide) has the formula: 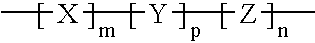
wherein:
m is a number from 0 to 1, inclusive;
p is a number from 0 to 1, inclusive;
n is a number from 0 to 1, inclusive, where:
m+p+n=1;
X has the chemical structure: 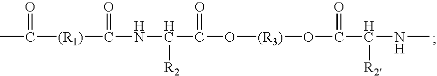
Y has the chemical structure: 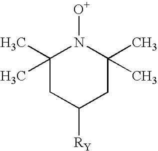
Z has the chemical structure: 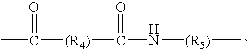
wherein:
R₁, R_{1′} and R₄ are independently selected from the group consisting of (1C-12C)alkyl and (2C-12C)alkenyl, with the proviso that if R₃ and R_{3′} are the same, then R₁ and R_{1′} are different;
R₂, R_{2′}, R_{2″} and R_{2′″}, are independently selected from the group consisting of hydrogen and (1C-4C)alkyl, wherein:
the alkyl group is optionally substituted with a moiety selected from the group consisting of —OH, —O(1C-4C)alkyl, —SH, —S(1C-4C)alkyl, —SeH, —COR₆, —NHC(NH)NH₂, imidazol-2-yl, imidazole-5-yl, indol-3-yl, phenyl, 4-hydroxyphenyl and 4-[(1C-4C)alkylO]phenyl, wherein:
R₆ is selected from the group consisting of —OH, —O(1C-4C)alkyl, —NH₂, —NH(1C-4C)alkyl,
—N(1C-4C)alkyl₁(1C-4C)alkyl₂, a stable nitroxide, and 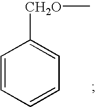
or
one or more of R₂, R_{2′}, R_{2″} and R_{2′″} may form a bridge between the carbon to which it is attached and the adjacent nitrogen, the bridge comprising —CH₂CH₂CH₂—;
R₃ and R_{3′} are independently selected from the group consisting of (1C-12C)alkyl, (2C-12C)alkenyl, (3C-8C)cycloalkyl and —(CH₂CH₂O)_{q}CH₂CH₂—, wherein q is an integer from 1 to 10, inclusive, with the proviso that if R₁ and R_{1′} are the same then R₃ and R_{3′} are different; and,
R₅ is selected from the group consisting of —CH(COR₆)CH₂S—, —CH(COR₆)CH₂O—, —CH(COR₆)(CH₂)₄NH—, —(CH₂)₄CH(COR₆)NH—,
—CH(COR₆)CH(CH₃)O—, 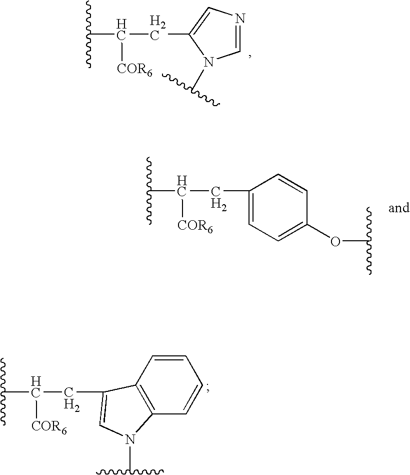
wherein at least one of R₂, R_{2′}, R_{2″}, R_{2′″} and R₅ comprises R₆, wherein R₆ comprises a stable nitroxide.

6. The implantable medical device of claim 5, wherein R₁, R_{1′} and R₄ are independently selected from the group consisting of —(CH₂)₄— and —(CH₂)₈—.

7. The implantable medical device of claim 6, wherein R₂, R_{2′}, R_{2″} and R_{2′″} are independently selected from the group consisting of —CH₃, —H₂CH₂NHC(NH)NH₂, —CH₂CONH₂, —CH₂COOH, —CH₂SH, —CH₂CH₂COOH, —CH₂CH₂CONH₂, —CH₂NH₂, 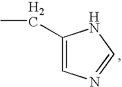
—CH(CH₃)CH₂CH₃.—CH₂CH(CH₃)₂, —(CH₂)₄NH₂, (CH₂)₂SCH₃, 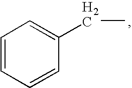
CH₂OH, —CH(CH₃)OH, 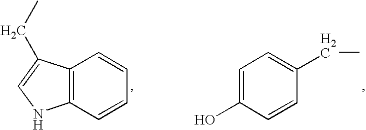
CH(CH₃)₂ and —CH₂CH₂CH₂—, wherein the second carbon is covalently bonded to the nitrogen adjacent to the carbon to which R₂ is bonded.

8. The implantable medical device of claim 7, wherein R₂, R_{2′}, R_{2″} and R_{2′″} are —CH₂CH(CH₃)₂.

9. The implantable medical device of claim 8, wherein:
R₃ is —(CH₂)₆—; and,
R₃ is

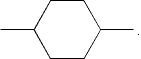

10. The implantable medical device of claim 9, wherein R₅ is —(CH₂)₄COR₆NH— and R₆ comprises a stable nitroxide.

11. The implantable medical device of claim 10 wherein the stable nitroxide is selected from the group consisting of: 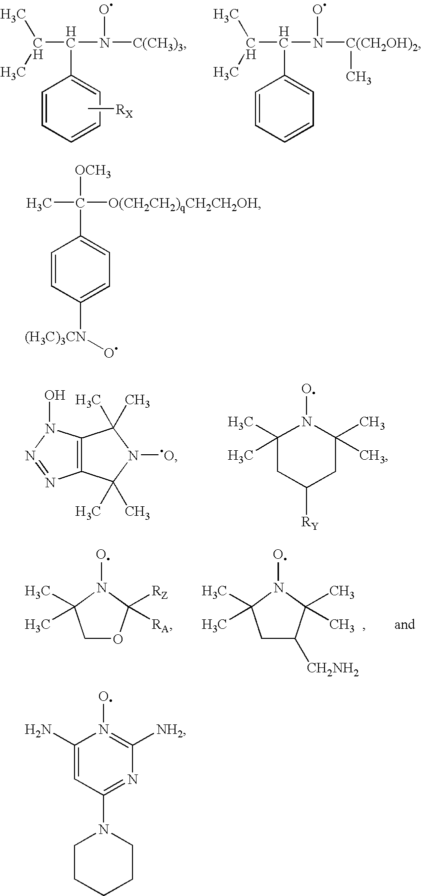
wherein Rₓ and R_{y} are selected from the group consisting of —NH₂, —OH and C(O)OH; R_{Z} is —(CH₂)_{b}C(O)OH and R_{A} is —(CH₂)_{b′}CH₃, where b and b′ are independently 1-16.

12. The implantable medical device of claim 11, wherein the stable nitroxide is 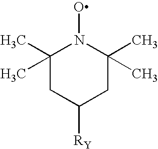
wherein R_{y} is —NH₂.

13. The implantable medical device of claim 1, wherein p=0.

14. The implantable medical device of claim 13, wherein R₁ and R₄ are independently selected from the group consisting of —(CH₂)₄— and —(CH₂)₈—.

15. The implantable medical device of claim 14, wherein R₂ and R_{2′} are independently selected from the group consisting of —CH₃, —CH₂CH₂NHC(NH)NH₂, —CH₂CONH₂, —CH₂COOH, —CH₂SH, —CH₂CH₂COOH, —CH₂CH₂CONH₂, —CH₂NH₂, 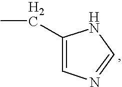
—CH(CH₃)CH₂CH₃.—CH₂CH(CH₃)₂, —(CH₂)₄NH₂, (CH₂)₂SCH₃ 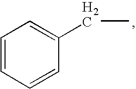
CH₂OH, —CH(CH₃)OH, 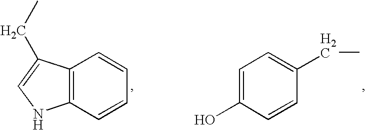
CH(CH₃)₂ and —CH₂CH₂CH₂—, wherein the second carbon is covalently bonded to the nitrogen adjacent to the carbon to which R₂ is bonded.

16. The implantable medical device of claim 15, wherein R₂ and R_{2′} are —CH₂CH(CH₃)₂.

17. The implantable medical device of claim 16, wherein R₃ is selected from the group consisting of —(CH₂)₃—, —(CH₂)₆— and —(CH₂CH₂O)_{q}CH₂CH₂—, wherein q is an integer from 1 to 10, inclusive.

18. The implantable medical device of claim 17, wherein q is 2.

19. The implantable medical device of claim 18, wherein R₅ is —(CH₂)₄CH(COR₆)NH— and R₆ comprises a stable nitroxide compound.

20. The implantable medical device of claim 19, wherein the stable nitroxide is selected from the group consisting of: 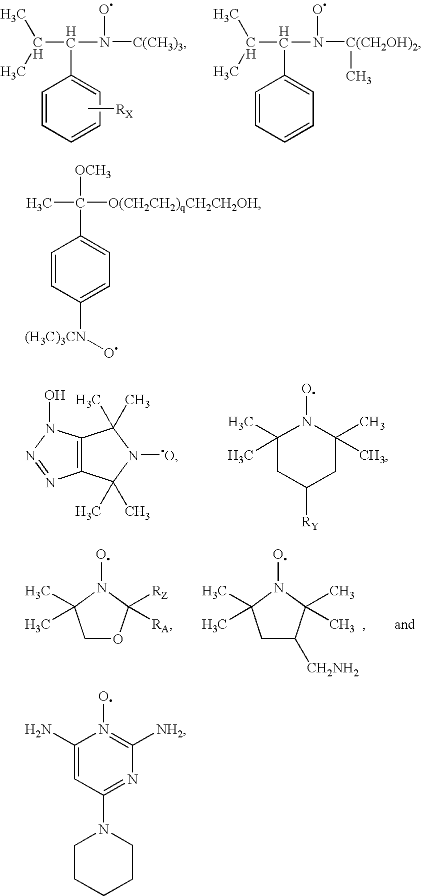
wherein Rₓ and R_{y} are selected from the group consisting of —NH₂, —OH and C(O)OH; R_{Z} is —(CH₂)_{b}C(O)OH and R_{A} is —(CH₂)_{b′ CH}₃, where b and b′ are independently 1-16.

21. The implantable medical device of claim 20, wherein the stable nitroxide is 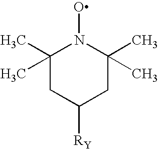
wherein R_{y} is —NH₂.

22. The implantable medical device of claim 5, wherein p and n are 0.

23. The implantable medical device of claim 22, wherein R₁ is selected from the group consisting of —(CH₂)₄— and —(CH₂)₈—.

24. The implantable medical device of claim 23, wherein R₂ and R_{2′} are independently selected from the group consisting of —CH₃, —H₂CH₂NHC(NH)NH₂, —CH₂CONH₂, —CH₂COOH, —CH₂SH, —CH₂CH₂COOH, —CH₂CH₂CONH₂, —CH₂NH₂, 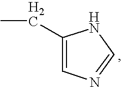
—CH(CH₃)CH₂CH₃.—CH₂CH(CH₃)₂, —(CH₂)₄NH₂, (CH₂)₂SCH₃, 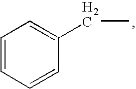
CH₂OH, —CH(CH₃)OH, 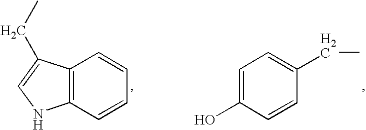
CH(CH₃)₂ and —CH₂CH₂CH₂—, wherein the second carbon is covalently bonded to the nitrogen adjacent to the carbon to which R₂ is bonded.

25. The implantable medical device of claim 24, wherein R₂ and R_{2′} are CH₂CH(CH₃)₂.

26. The implantable medical device of claim 25, wherein R₃ is selected from the group consisting of —(CH₂)₃—, —(CH₂)₆— and —(CH₂CH₂O)_{q}CH₂CH₂—, wherein q is an integer from 1 to 10, inclusive.

27. The implantable medical device of claim 26, wherein q is 2.

28. The implantable medical device of claim 27, wherein R₅ is —(CH₂)₄CH(COR₆)NH—, wherein R₆ comprises a stable nitroxide.

29. The implantable medical device of claim 28, wherein the stable nitroxide is selected from the group consisting of: 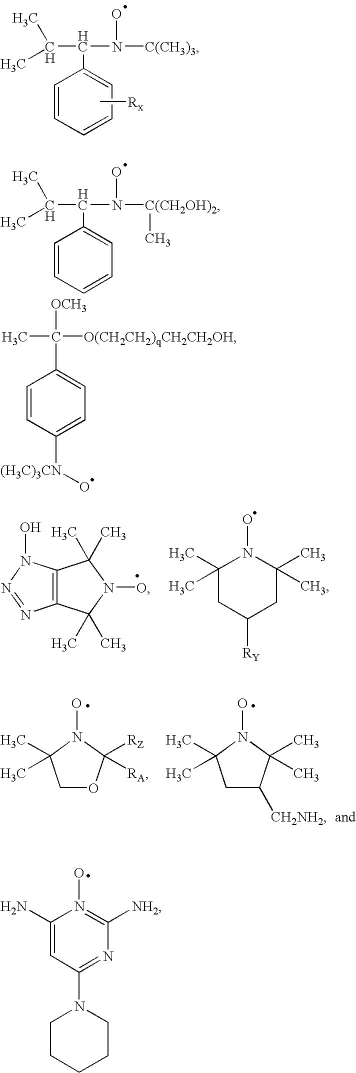
wherein Rₓ and R_{y} are selected from the group consisting of —NH₂, —OH and C(O)OH; R_{Z} is —(CH₂)_{b}C(O)OH and R_{A} is —(CH₂)_{b′}CH₃, where b and b′ are independently 1-16.

30. The implantable medical device of claim 29, wherein the stable nitroxide is 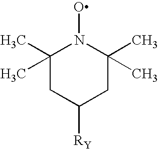
wherein R_{y} is —NH₂.

31. The implantable medical device of claim 1, wherein the therapeutic agent is everolimus.

32. The implantable medical device of claim 1, wherein the device is a stent.

33. A method comprising:
providing an implantable medical device, wherein the device comprises:
a device body;
an optional primer layer disposed over the device body;
a drug reservoir layer disposed over the device body or over the primer layer, if opted; the drug reservoir layer comprising one or more therapeutic agents;
an optional rate-controlling layer disposed over the drug reservoir layer;
an optional topcoat layer disposed over the drug reservoir layer or the rate-controlling layer, if opted; and,
a stable nitroxide, wherein:
the stable nitroxide is contained in the drug reservoir layer, in the rate-controlling layer, in the topcoat layer, in a separate layer disposed between the drug reservoir layer and a source of free radicals generated to sterilize the device; and,
sterilizing the device using a free-radical generating sterilization method.

34. The method of claim 33, wherein the free-radical generating sterilization method is e-beam sterilization.

35. The method of claim 33, wherein the stable nitroxide is a polymer-bound stable nitroxide.

36. The method of claim 35, wherein the polymer is a poly(ester-amide).

37. The method of claim 36, wherein the poly(ester-amide) has the formula:

38. The method of claim 37, wherein the stable nitroxide is selected from the group consisting of: 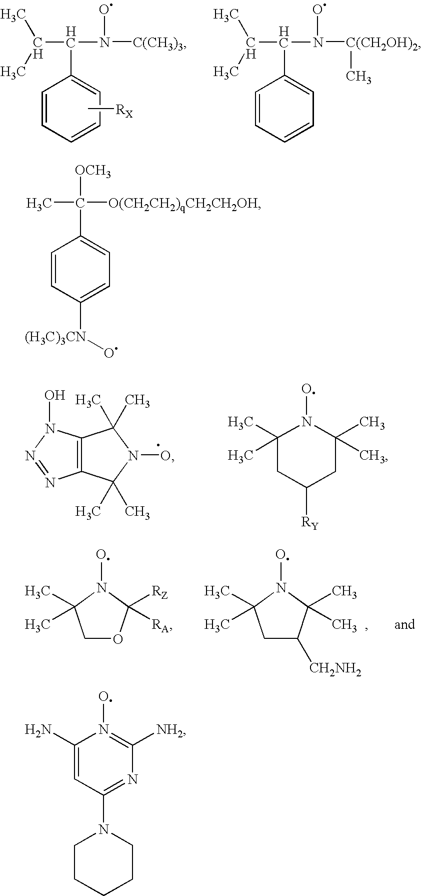
wherein Rₓ and R_{y} are selected from the group consisting of —NH₂, —OH and C(O)OH; R_{Z} is —(CH₂)_{b}C(O)OH and R_{A} is —(CH₂)_{b′}CH₃, where b and b′ are independently 1-16.

39. The method of claim 38, wherein the stable nitroxide is 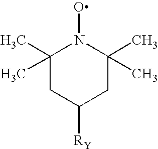
wherein R_{y} is —NH₂.

40. The method of claim 33, wherein the implantable medical device is a stent.

41. The method of claim 33, wherein the therapeutic agent is everolimus.
